(19) United States
(12) Patent Application Publication
(10) Pub. No.: US 2024/0398686 A1
(43) Pub. Date: Dec. 5, 2024

(54) EFFERVESCENT CLEANSING POWDER COMPOSITION

(71) Applicant: Conopco, Inc., d/b/a UNILEVER, Englewood Cliffs, NJ (US)

(72) Inventors: Hasiba BEKTO, Bristol, CT (US); Jenna Christine DOUTHIT, Derby, CT (US); Kayla Marie KEMLER, Milford, CT (US)

(73) Assignee: Conopco, Inc., d/b/a UNILEVER, Englewood Cliffs, NJ (US)

(21) Appl. No.: 18/695,864

(22) PCT Filed: Aug. 30, 2022
(86) PCT No.: PCT/EP2022/074093

§ 371 Date: Mar. 27, 2024

(30) Foreign Application Priority Data
Oct. 15, 2021 (EP) 21202863.3

(51) Int. Cl. A61K 8/73; A61K 8/365; A61Q 19/10

(52) U.S. Cl. CPC A61K 8/731; A61K 8/365; A61Q 19/10; A61K 2800/22; A61K 2800/28; A61K 2800/805

(57) ABSTRACT

The present invention is directed to an effervescent cleansing powder composition that comprises an effervescent system, a solid surfactant system, and a stabilizing system comprising apolysaccharide builder and a salt of carboxylic acid, and optionally a viscosity builder. The cleansing powder composition is mild during use, generates consumer-desirable lathering characteristics, and remains free-flowing under high humidity and/or temperature conditions.

### FIELD OF THE INVENTION

The present invention is directed to an effervescent cleansing powder composition. More particularly, the cleansing powder composition comprises an effervescent system, a solid surfactant system, a stabilizing system comprising a polysaccharide builder and a carboxylic acid salt, and optionally a viscosity builder. The cleansing powder composition is mild during use and generates consumer-desirable lathering characteristics. Such a cleansing powder composition unexpectedly remains free-flowing under high humidity and/or high temperature conditions, is convenient to evacuate from packaging and provides superior self-lather during use.

### BACKGROUND OF THE INVENTION

Liquid based cleansing compositions, such as shampoos, body washes and hard surface cleaners are common and enjoyed by many consumers. These conventional compositions typically have water as the predominant ingredient, and they are often sold in plastic bottles, plastic pump containers and tubes. The compositions are conventionally formulated to have a viscosity that is customary for consumer use and easy for evacuation from the packaging they are sold in.

It is often publicized that the world's oceans will soon have more plastic than fish. Given environmental concerns and the desire for consumers and conscious companies to do more for the planet, there is a strong desire to use less plastic when selling products, including consumer products. In view of this, efforts have been made to sell product in concentrate form, and therefore, to ship product that comprises less water. The difficulty with concentrates is that consumers often find concentrates to be difficult to dispense, as many are not humidity- and/or heat-proof, thereby resulting in undesirable agglomeration. This is of particular importance when the concentrate is intended to be used in a steamy environment such as a residential bathroom. Other complaints include that the end use product is of a water-thin viscosity (i.e., does not thicken upon hydration and therefore possesses undesirable viscosity), is not homogenous, or produces insufficient lather or requires performing further work to obtain a desirable lather.

As to the hydrated product originating from concentrates, common complaints include that the product is not homogeneous after adding water and/or of undesirable viscosity. Moreover, concentrates are not typically used to prepare single use amounts (or dosages) of end use consumer product.

It is of increasing interest to develop a cleansing powder composition that is easy to carry and hydrate, and that results in consumer-desirable lathering with limited shear and water. It is also of increasing interest to have a cleansing powder composition that can be easily used to prepare single use applications of end use consumer wash product. This invention, therefore, is directed to an effervescent cleansing powder composition that comprises a solid surfactant system comprising an isethionate; an effervescent system comprising a dry acidic material and a dry alkaline material; a stabilizing system comprising a polysaccharide builder and a salt of carboxylic acid; and optionally a viscosity builder. The cleansing powder composition is gentle in use and is self-foaming. Such a cleansing powder composition unexpectedly does not aggregate, is convenient to evacuate from packaging and is easy to hydrate for a single use application.

### Additional Information

Efforts have been disclosed for making powdered compositions exhibiting effervescence.

U.S. Pat. No. 6,506,713 discloses a cosmetic cleansing article including an effervescent cleanser composition comprising an acid material and an alkaline material contained within a sachet having at least one water-permeable wall.

U.S. Pat. Nos. 6,063,390, 6,217,854, 6,610,312, 6,723,330, 6,878,380 report articles for cleansing body surfaces including an effervescent cleansing composition capable of creating a foam upon contact with water, and a pouch for housing the composition. The cleansing composition comprises an alkaline material and an acid.

U.S. Pat. Nos. 7,507,396 and 7,919,126 disclose an effervescent composition and a method of making an effervescent composition including a viscous component and a free-flowing granulation.

U.S. Published Patent Application No. 2014/0020701 relates to an anhydrous topical composition comprising a) a base compound of a carbonate or bicarbonate salt of an alkali or alkaline earth metal; b) an acid compound; and c) an anhydrous carrier, and such a composition effervesces when exposed to water.

PCT application WO 2020/113484 relates to a solid cosmetic composition in form of a tablet or powder containing at least one solid anionic surfactant, an effervescent system developing gas and a disintegration system.

A number of commercially available powder cleansing compositions exist on the market, such as Bioré® Baking Soda Cleansing Scrub, DHC Face Wash Powder, Susteau Moondust Hair Wash, and OLAY® Exfoliating Face & Body Powder in Pitaya & Cranberry.

None of the additional information describes a cleansing powder application as described and claimed in the present application.

### SUMMARY OF THE INVENTION

In a first aspect, disclosed herein is a cleansing powder composition comprising:
a) a solid surfactant system comprising an isethionate, taurate, or a mixture thereof;
b) an effervescent system comprising a dry acidic material having an acid dissociation constant (pKₐ) from 2 to 12, preferably, 2 to 8, and, most preferably, 2 to 6, and a logP value less than 0, preferably, from −2 to 0.01, and, most preferably, −1.5 to −0.05, and a dry alkaline material;
c) a stabilizing system comprising a polysaccharide builder and a salt of carboxylic acid; and
d) optionally a viscosity builder.

In a second aspect, disclosed is a method of cleaning with the cleansing powder composition of the first aspect by hydrating the composition with shear to produce an end use wash composition and contacting skin, hair, nails or an inanimate object with the end use wash composition.

In a third aspect, disclosed herein is directed to the use of a composition comprising:
a) a solid surfactant system comprising an isethionate, taurate, or a mixture thereof;
b) an effervescent system comprising a dry acidic material having an acid dissociation constant (pKₐ) from 2 to 12, preferably, 2 to 8, and, most preferably, 2 to 6, and a logP value less than 0, preferably, from −2 to 0, and, most preferably, −1.5 to −0.01, and a dry alkaline material;
c) a stabilizing system comprising a polysaccharide builder and a salt of carboxylic acid; and
d) optionally a viscosity builder.
to clean skin, hair, nails or an inanimate object.

Cleansing powder composition as used herein means a composition that is hydratable and suitable to clean skin, hair, nails or an inanimate object. Solid surfactant system comprising an isethionate means the total source of surfactant in the cleansing composition powder may comprise solely isethionate or taurate, or isethionate and/or taurate in combination with other desirable surfactants commonly used in the field. In an embodiment of the invention, isethionate makes up from 50 to 90% by weight of the surfactant in the surfactant system. In another embodiment of the invention, isethionate makes up 100% by weight of the total surfactant in the system. In yet another aspect, taurate makes up from 50 to 90% by weight of the surfactant in the system. In still another aspect, taurate makes up 100% by weight of the total surfactant in the system. Polysaccharide builder refers to a carbohydrate or starchy material that does not swell upon contact and/or dilution with water. As used herein, a viscosity builder is a viscosity modifier or structurant that will instantly swell upon contact and/or dilution with water and thereby builds (i.e., contributes to) the viscosity of the end use wash composition and/or builds smooth sensory benefits at room temperature (i.e., 25° C.). The term “acid” or “acidic material” is intended to refer to any substance which when dissolved in deionized water at 1% concentration will have a pH of less than 7, preferably less than 6.5, more preferably less than 5, while the term “base” or “alkaline material” is intended to refer to any substance which can generate a gas such as carbon dioxide, nitrogen, or oxygen (i.e., effervesce) when contacted with water and acid. As used herein, the terms “dry,” “solid” or “anhydrous” are to be used interchangeably and means essentially free of water (i.e., the presence of 0 to 5% water, preferably, 0.01 to 3%, and, optimally, 0.05 to 1% water by weight of the specified ingredient). Water of hydration is not considered to be water for purposes of the anhydrous definition, but it is preferred to minimize, preferably to eliminate any water of hydration. “logP,” as used herein, refers to a conventional measure of lipophilicity, specifically meaning the logarithmic value of the partition coefficient of the desired compound. A lower logP means that the compound has a higher affinity for an aqueous phase (i.e., is more hydrophilic) while a higher logP means that the compound has a higher affinity for an oil phase (i.e., is more lipophilic). A logP of 0 means the compound is equally partitioned between the aqueous and oil phases. “Effervescent,” as used herein, refers to an instantaneous formation of gas bubbles (i.e., foaming or fizzing) from a chemical reaction in a liquid and the term is used interchangeably herein with “self-foaming.” Foam volume refers to the volume of foam generated when the cleansing powder composition is hydrated with water. By extension, peak foam volume as used herein is meant to describe the maximum volume of foam produced as a result of the aforementioned effervescent reaction during the course of the reaction.

Skin, as used herein, includes skin on the feet, face, neck, chest, arms (including under arms), hands, legs, buttocks, back and scalp. Inanimate object is meant to include a hard surface like a counter, glass, an appliance door, vehicle and a porcelain fixture such as a dish, mug, sink, tub or toilet. Inanimate object is also meant to include materials comprising nylon, cotton and/or polyester (e.g., clothes, curtains, shirts, rugs, sneakers, upholstery), and fruits and vegetables that are typically washed before consumption. The cleansing powder composition after hydration (i.e., combining with water) is herein referred to as an end use (wash) composition that is interchangeable with “hydrated product.” Therefore, the cleansing powder composition itself is a precursor to an end use composition. The end use wash composition includes shampoos for hair washing, body washes, automobile wash products as well as home care wash compositions like hard surface and window cleaners, toilet bowl cleaners and laundry detergents. In one embodiment, the end use wash composition of the present invention is a hand, body, face, scalp and/or nail wash composition. In another embodiment, the end use wash composition is a shampoo (i.e., hair washing) composition. In still another embodiment, the end use wash composition is a home care composition. Powder flow, as used herein, means easy to shake out of a shaker, where powder clumping and static cohesion are not visually observed. Similarly, as used herein, the term “free-flowing” is meant to describe a powder that moves in a continuous steady flow.

Unless explicitly stated otherwise, all ranges described herein are meant to include all ranges subsumed therein. The term comprises is meant to encompass the terms consisting essentially of and consisting of. For the avoidance of doubt, a composition comprising a solid surfactant system, dry acidic material, dry alkaline material, polysaccharide builder, salt of carboxylic acid, and optionally a viscosity builder is meant to include a composition consisting essentially of the same and a composition consisting of the same. As to the percentages used herein, the same are meant to be by weight of ingredient, (e.g., not including any water it may be supplied with) unless noted otherwise. Except in the operating and comparative examples, or where otherwise explicitly indicated, all numbers used in this description indicating amounts, or ratios of materials and/or use thereof are to be understood as modified by the word “about”.

### DETAILED DESCRIPTION OF THE INVENTION

Cleansing powder compositions disclosed herein comprise an effervescent system having a dry acidic material and a dry alkaline material. As to the acidic material, desirable for this purpose are any acids present in dry solid form. Also desirable are any acids having an acid dissociation constant (pKₐ) from 2 to 12, preferably, 2 to 8, and, most preferably, 2 to 6, and a logP value less than 0, preferably, from −2 to −0.05, and, most preferably, −1.75 to −0.01. Preferably, such acids are C₂-C₂₀ organic mono- and poly-carboxylic acids, more preferably, alpha- and beta-hydroxycarboxylic acids. Illustrative but nonlimiting examples include C₂-C₂₀ organophosphorus acids such as phytic acid; peroxides such as hydrogen peroxide; hydroxycarboxylic acids such as glutaric, succinic, tartaric, malic, lactic, and citric acids as well as acid-forming lactoses such as gluconolactone and glucarolactone; and mixtures thereof. Illustrative examples of such acidic materials with desirable pKₐ and logP values include citric acid (pKₐ=2.9, 4.3, 5.2; logP=−1.6), lactic acid (pKₐ=3.8; logP=−0.7), acetic acid (pKₐ=4.8; logP=−0.3), glucanolactone (pKₐ=11.6; logP=−2.2), and hydrogen peroxide (pKₐ=11.6; logP=−0.4). It is especially preferred that the dry acidic material comprises citric acid. Also desirable as acidic material may be encapsulated and/or coated acids, an example of which includes CITROCOAT® N, which is citric acid coated by a monosodium citrate shell and which is made commercially available from the supplier Jungbunzlauer. The acidic material is present in amounts of 5 to 35%, preferably, 7 to 32%, optimally, 10 to 30% by weight of the cleansing powder composition. In another preferred aspect, the cleansing powder composition comprises 12 to 28% acidic material by weight of the cleansing powder composition.

Alkaline material desirable for use in the inventive cleansing powder composition are anhydrous, metal salts of carbonates and bicarbonates, alkaline peroxides (e.g., sodium perborate and sodium percarbonate) and azides (e.g., sodium azide). It is preferred that the alkaline material comprises sodium bicarbonate, potassium bicarbonate, sodium sesquicarbonate, or a mixture thereof. The alkaline material is present in amounts of 5 to 35%, preferably, 7 to 32%, optimally, 10 to 30% by weight of the cleansing powder composition. In another aspect, the cleansing powder composition comprises 12 to 28% alkaline material by weight of the cleansing powder composition.

In an aspect of the disclosed cleansing powder composition, the weight ratio of dry acidic material to dry alkaline material in the cleansing powder composition is 30:70 to 70:30, preferably, 40:60 to 60:40, and, more preferably, 45:55 to 55:45.

As to the isethionate used in the solid surfactant system of the cleansing powder composition, the same is limited only to the extent that it is one suitable for use in a consumer product and that it is available for use in solid form. The cleansing powder composition may contain C₈-C₁₈ acyl isethionates. These esters are prepared by a reaction between alkali metal isethionate with mixed aliphatic fatty acids having from 6 to 18 carbon atoms and an iodine value of less than 20. At least 75% of the mixed fatty acids have from 12 to 18 carbon atoms and up to 25% have from 6 to 10 carbon atoms. Isethionates may be present in an amount of 5 to 40%, preferably, 10 to 35%, and, more preferably, 12 to 30% by weight of the cleansing powder composition. In another aspect, isethionate may be included in an amount of 15 to 30% by weight of the cleansing powder composition. Isethionate may comprise from 50 to 90% by weight of the surfactant in the total surfactant system. In yet another embodiment of the invention, isethionate makes up 100% by weight of the total surfactant in the system.

The acyl isethionate may be an alkoxylated isethionate such as is described in Ilardi et al., U.S. Pat. No. 5,393,466, entitled “Fatty Acid Esters of Polyalkoxylated isethonic acid; issued Feb. 28, 1995; hereby incorporated by reference. This compound has the general formula:

R¹C—(O)O—C(X)H—C(Y)H—(OCH₂—CH₂)ₘ—SO₃M

wherein R¹ is an alkyl group having 8 to 18 carbons, m is an integer from 1 to 4, X and Y are each independently hydrogen or an alkyl group having 1 to 4 carbons and M is a solubilizing cation such as hydrogen, sodium, potassium, ammonium, or substituted ammonium. Illustrative examples of the isethionate surfactant that may be used in the disclosed cleansing powder composition herein include, for example, sodium lauroyl isethionate and sodium cocoyl isethionate. The solid surfactant system having the aforementioned isethionate may comprise solely of an isethionate or an isethionate in combination with other desirable surfactants commonly used in the field.

As to the taurate used in the surfactant system of the cleansing powder composition, the same is limited only to the extent that it is one suitable for use in a consumer product. Illustrative examples of the taurate surfactant that may be used in the cleansing powder composition of the invention include, for example, those which are acylamides of taurine or N-methyltaurine, and salts thereof. For example, taurates suitable for use are acyl taurates represented by the general formulae:

R²C(O)N(R³)(CH₂)_{y}SO₃M (I), and

R²C(O)N(R³)CH₂CH₂SO₃M (II),

where R² is C₆ to C₃₀, more particularly, C₆ to C₂₄ alkyl, y is 2 or 3, R³ is hydrogen or methyl, and M is hydrogen or a solubilizing cation such as, for example, hydrogen, ammonium, alkali metal cation, a lower C₁ to C₄, alkanol ammonium cation and/or a basic amino acid cation. In one embodiment, R² is C₈ to C₁₈ alkyl. In another embodiment at least half of the R² groups are C₈-C₁₈ alkyl. In still another embodiment at least half of the R² groups are C₁₀ to C₁₄ alkyl. R² may be saturated or unsaturated. In yet another embodiment R³ is methyl.

Illustrative acyl taurates that may be used in the surfactant system of the inventive cleansing powder composition include, for example, taurates commonly known as sodium methyl lauroyl taurate, sodium methyl myristoyl taurate, sodium methyl cocoyl taurate, sodium methyl oleoyl taurate, sodium cocoyl taurate, mixtures thereof or the like. In an embodiment, the taurate used is sodium methyl lauroyl taurate. Taurates may be present in an amount of 5 to 40%, preferably, 10 to 35%, and, more preferably, 12 to 30% by weight of the cleansing powder composition. In another aspect, taurate may be included in an amount of 15 to 30% by weight of the cleansing powder composition. Taurate may comprise from 50 to 90% by weight of the surfactant in the total surfactant system. In another aspect, taurate makes up 100% by weight of the total surfactant in the system.

Other anionic surfactants may be used in the disclosed cleansing powder composition in addition to isethionate, so long as they are available in solid form. These additional anionic surfactants may include alkyl sulfosuccinates (including mono- and dialkyl, e.g., C₆-C₂₂ sulfosuccinates); alkyl and acyl sarcosinates, sulfoacetates, C₈-C₂₂ alkyl phosphates and phosphonates, alkyl phosphate esters and alkoxyl alkyl phosphate esters, acyl lactates, C₈-C₂₂ monoalkyl succinates and maleates, sulphoacetates, alkyl glucosides and acyl isethionates, acyl glutamates, glycinates and the like.

Sulfosuccinates may be monoalkyl sulfosuccinates having the formula:

R⁴OC(O)CH₂CH(SO₃M)CO₂M;

and amide-MEA sulfosuccinates of the formula:

R⁴CONHCH₂CH₂OC(O)CH₂CH(SO₃M)CO₂M

wherein R⁴ ranges from C₈-C₂₂ alkyl.

Sarcosinates are generally indicated by the formula:

R⁵CON(CH₃)CH₂CO₂M,

wherein R⁵ ranges from C₈-C₂₀ alkyl.

Glycinates generally have the formula:

R⁶CONR⁷CH₂CO₂M,

wherein R⁶ is a C₈-C₂₄ alkyl, R⁷ is hydrogen or CH₃.

Acyl glutamates generally have the formula:

R⁸CONH(CHCO₂H)CH₂CH₂CO₂M or

R⁸CONH(CHCO₂M)CH₂CH₂CO₂H,

wherein R⁸ is a C₈-C₂₀ alkyl or alkenyl.

M is a solubilizing cation as previously described.

In an aspect of the cleansing powder composition, the additional anionic surfactant used is 2-acrylamido-2-methylpropane sulfonic acid, ammonium lauryl sulfate, ammonium perfluorononanoate, potassium lauryl sulfate, sodium alkyl sulfate, sodium dodecyl sulfate, sodium laurate, sodium laureth sulfate, sodium lauroyl sarcosinate, sodium stearate, sodium sulfosuccinate esters, sodium lauroyl glutamate, sodium myristoyl glutamate, sodium stearoyl glutamate, sodium cocoyl glutamate, potassium myristoyl glutamate, sodium cocoyl glycinate, potassium cocoyl glycinate, or a combination thereof. Such anionic surfactants are commercially available from suppliers like Galaxy Surfactants, Clariant, Sino Lion, Stepan Company, and Innospec. Preferably, the additional anionic surfactant used is a sodium alkyl sulfate like sodium lauryl sulfate, an acyl glutamate, a glycinate, or a mixture thereof.

Optionally, amphoteric surfactants can be included in the cleansing powder compositions disclosed herein, so long as they are available in solid form. Amphoteric surfactants (which depending on pH can be zwitterionic) include sodium acyl amphoacetates, sodium acyl amphopropionates, disodium acyl amphodiacetates and disodium acyl amphodipropionates where the acyl (i.e., alkanoyl group) can comprise a C₇-C₁₈ alkyl portion. Illustrative examples of amphoteric surfactants include sodium lauroamphoacetate, sodium cocoamphoacetate, sodium lauroamphoacetate, or a combination thereof. In one aspect, the cleansing powder composition comprises less than 2%, preferably, less than 1% amphoteric surfactant by weight of the cleansing powder composition. In another aspect, cleansing powder composition comprises 0.001 to less than 1%, preferably, 0.001 to 0.85%, more preferably, 0.001 to 0.75%, and, still more preferably, 0.005 to 0.5% amphoteric surfactant by weight of the cleansing powder composition. In yet another aspect, the cleansing powder composition comprises 0% amphoteric surfactant by weight of the cleansing powder composition.

As to the solid zwitterionic surfactants employed in the present cleansing powder composition, such surfactants include at least one acid group. Such an acid group may be a carboxylic or a sulphonic acid group. They often include quaternary nitrogen, and therefore, can be quaternary amino acids. They should generally include an alkyl or alkenyl group of 7 to 18 carbon atoms and generally comply with an overall structural formula:

R⁹—[—C(O)—NH(CH₂)_{q}—]ᵣ—N⁺(R¹⁰)(R¹¹)-A-B,

where R⁹ is alkyl or alkenyl of 7 to 18 carbon atoms; R¹⁰ and R¹¹ are each independently alkyl, hydroxyalkyl or carboxyalkyl of 1 to 3 carbon atoms; q is 2 to 4; r is 0 to 1; A is alkylene of 1 to 3 carbon atoms optionally substituted with hydroxyl, and B is —CO₂— or —SO₃—.

Desirable zwitterionic surfactants for use in the cleansing powder composition disclosed herein and within the above general formula include simple betaines of formula:

R⁹—N⁺(R¹⁰)(R¹¹)—CH₂CO₂⁻

and amido betaines of formula:

R⁹—CONH(CH₂)—N⁺(R¹⁰)(R¹¹)—CH₂CO₂⁻,

where t is 2 or 3.

In both formulae R⁹, R¹⁰ and R¹¹ are as defined previously. R⁹ may, in particular, be a mixture of C₁₂ and C₁₄ alkyl groups derived from coconut oil so that at least half, preferably, at least three quarters of the groups R⁹ have 10 to 14 carbon atoms. R¹⁰ and R¹¹ are preferably methyl.

A further possibility is that the zwitterionic surfactant is a sulphobetaine of formula:

R⁹—N⁺(R¹⁰)(R¹¹)—(CH₂)₃SO₃⁻; or

R⁹—CONH(CH₂)ᵤ—N⁺(R¹⁰)(R¹¹)—(CH₂)₃SO₃⁻,

where u is 2 or 3, or variants of these in which —(CH₂)₃SO₃⁻ is replaced by —CH₂C(OH)(H)CH₂SO₃⁻.

In these formulae, R⁹, R¹⁰ and R¹¹ are as previously defined.

Illustrative examples of the zwitterionic surfactants desirable for use include betaines such as lauryl betaine, betaine citrate, cocodimethyl carboxymethyl betaine, cocoamidopropyl betaine, coco alkyldimethyl betaine, and laurylamidopropyl betaine. An additional zwitterionic surfactant desirable for use includes cocoamidopropyl sultaine, for example, cocamidopropyl hydroxysultaine. Preferred zwitterionic surfactants include lauryl betaine, betaine citrate, sodium hydroxymethylglycinate, (carboxymethyl) dimethyl-3-[(1-oxododecyl) amino] propylammonium hydroxide, coco alkyldimethyl betaine, (carboxymethyl) dimethyloleylammonium hydroxide, cocoamidopropyl betaine, (carboxymethyl) dimethyloleylammonium hydroxide, cocoamidopropyl betaine, (carboxylatomethyl) dimethyl(octadecyl) ammonium, cocamidopropyl hydroxysultaine, or a combination thereof. Such surfactants are made commercially available from suppliers like Stepan Company, Solvay, Evonik and the like and it is within the scope of the cleansing powder compositions disclosed herein to employ mixtures of the aforementioned surfactants.

Nonionic surfactants may optionally be used in the cleansing powder composition. When used, nonionic surfactants are typically used at levels as low as 0.01, 0.1, 1 or 2% by weight and at levels as high as 6, 8, 10 or 12% by weight. The nonionic surfactants which may be used include in particular the reaction products of compounds having a hydrophobic group and a reactive hydrogen atom, for example aliphatic alcohols, acids, amides or alkylphenols with alkylene oxides, especially ethylene oxide either alone or with propylene oxide. Specific nonionic surfactant compounds are alkyl (C₆-C₂₂) phenols, ethylene oxide condensates, the condensation products of aliphatic (C₈-C₁₈) primary or secondary linear or branched alcohols with ethylene oxide, and products made by condensation of ethylene oxide with the reaction products of propylene oxide and ethylenediamine. Other nonionic surfactants include long chain tertiary amine oxides, long chain tertiary phosphine oxides, dialkyl sulphoxides, and the like.

In an aspect, nonionic surfactants can include fatty acid/alcohol ethoxylates having the following structures a) HOCH₂ (CH₂)ₛ(CH₂CH₂O)_{c} H or b) HOOC(CH₂)ᵥ(CH₂CH₂O)_{d} H; where s and v are each independently an integer up to 18; and c and d are each independently an integer from 1 or greater. In an aspect, s and v can be each independently 6 to 18; and c and d can be each independently 1 to 30. Other options for nonionic surfactants include those having the formula HOOC(CH₂)ᵢ—CH═CH—(CH₂)^{k}(CH₂CH₂O)_{z} H, where i, k are each independently 5 to 15; and z is 5 to 50. In another aspect, i and k are each independently 6 to 12; and z is 15 to 35.

The nonionic surfactant may also include a sugar amide, such as a polysaccharide amide. Specifically, the surfactant may be one of the lactobionamides described in U.S. Pat. No. 5,389,279 to Au et al., entitled “Compositions Comprising Nonionic Glycolipid Surfactants issued Feb. 14, 1995; which is hereby incorporated by reference or it may be one of the sugar amides described in U.S. Pat. No. 5,009,814 to Kelkenberg, titled “Use of N-Poly Hydroxyalkyl Fatty Acid Amides as Thickening Agents for Liquid Aqueous Surfactant Systems” issued Apr. 23, 1991; hereby incorporated into the subject application by reference.

Illustrative examples of nonionic surfactants that can optionally be used in the cleansing powder compositions disclosed herein include, but are not limited to, polyglycoside, cetyl alcohol, decyl glucoside, lauryl glucoside, octaethylene glycol monododecyl ether, n-octyl beta-d-thioglucopyranoside, octyl glucoside, oleyl alcohol, polysorbate, sorbitan, stearyl alcohol, or a combination thereof.

In an aspect, cationic surfactants may optionally be used in the cleansing powder composition of the present application.

One class of cationic surfactants includes heterocyclic ammonium salts such as cetyl or stearyl pyridinium chloride, alkyl amidoethyl pyrrylinodium methyl sulfate, and lapyrium chloride.

Tetra alkyl ammonium salts are another useful class of cationic surfactants for use. Examples include cetyl or stearyl trimethyl ammonium chloride or bromide; hydrogenated palm or tallow trimethylammonium halides; behenyl trimethyl ammonium halides or methyl sulfates; decyl isononyl dimethyl ammonium halides; ditallow (or distearyl) dimethyl ammonium halides, and behenyl dimethyl ammonium chloride.

Still other types of cationic surfactants that may be used are the various ethoxylated quaternary amines and ester quats. Examples include PEG-5 stearyl ammonium lactate (e.g., Genamin KSL manufactured by Clariant), PEG-2 coco ammonium chloride, PEG-15 hydrogenated tallow ammonium chloride, PEG 15 stearyl ammonium chloride, dipalmitoyl ethyl methyl ammonium chloride, dipalmitoyl hydroxyethyl methyl sulfate, and stearyl amidopropyl dimethylamine lactate.

Still other useful cationic surfactants include quaternized hydrolysates of silk, wheat, and keratin proteins, and it is within the scope of the cleansing powder composition to use mixtures of the aforementioned cationic surfactants.

If used, cationic surfactants will make up no more than 5% by weight of the cleansing powder composition. When present, cationic surfactants typically make up from 0.01 to 2%, and more typically, from 0.1 to 1% by weight of the cleansing powder composition, including all ranges subsumed therein.

Particularly preferred surfactants in addition to isethionate and taurate for use in the present cleansing powder compositions include wherein the surfactant is cocoamidopropyl betaine, sodium lauryl sulfate, sodium lauroyl glutamate, sodium cocoyl glycinate, potassium cocoyl glycinate, disodium lauryl sulfosuccinate, or a combination thereof.

The solid surfactant system used in the cleansing powder composition of this invention typically makes up a total of 5 to 40%, preferably, 10 to 35%, and, more preferably, 12 to 30% by weight of the cleansing powder composition. In one aspect, the surfactant system may be included in an amount of 15 to 30% by weight of the cleansing powder composition. In another aspect, the surfactant system makes up from 22 to 28% by weight of the cleansing powder composition.

The disclosed cleansing powder composition also comprises a stabilizing system comprising a polysaccharide builder and a salt of carboxylic acid. Such a stabilizing system has been determined to unexpectedly contribute to an enhanced stability in conditions characterized by high humidity and/or high temperature. A representative condition of high humidity is 80% relative humidity (RH) at 25° C. and a representative condition of high humidity and high temperature is 75% RH at 40° C. Without wishing to be bound by theory, it is believed that such a system helps to protect powdered materials in the cleansing powder composition from caking and settling under high humidity and/or high temperature conditions.

The stabilizing system disclosed herein comprises a salt of a carboxylic acid. In one aspect, the carboxylic acid is citric acid. It is preferred that the salt of citric acid is an alkali citrate, alkaline citrate, or a mixture thereof. Illustrative but non-limiting examples include tricalcium citrate, trimagnesium citrate, trisodium citrate, monosodium citrate, and mixtures thereof. Tricalcium citrate, trimagnesium citrate, and trisodium citrate are all made commercially available from suppliers such as Jungbunzlauer. Monosodium citrate may be available through the supplier Sigma-Aldrich. The cleansing powder composition comprises from 1 to 20%, preferably, 1.5 to 15%, and, more preferably, 2 to 10% by weight carboxylic acid salt. Particularly preferred, the cleansing powder composition comprises from 2.5 to 8% by weight carboxylic acid salt. In lieu of carboxylic acid salt, it has also been found that talc, silica and derivatives thereof, or a mixture thereof function similarly in the stabilizing system disclosed and such compounds may also be used in conjunction with a salt of carboxylic acid.

Polysaccharide builder used is limited only to the extent that the same is suitable for use in a topical composition. These includes include fibers, starches, and non-modified cellulose (e.g., cellulose microfibrils, cellulose nanocrystals or microcrystalline cellulose). Representative of the starches are chemically modified starches such as sodium hydroxypropyl starch phosphate and aluminum starch octenylsuccinate and non-modified starches such as corn starch, rice starch, tapioca starch, and maltodextrin. It is preferred that the polysaccharide builder comprises corn starch, tapioca starch, maltodextrin, microcrystalline cellulose, or a mixture thereof. As to polysaccharide builder used in the cleansing powder composition, 8 to 60%, preferably, 10 to 50%, and, more preferably, 12 to 40% by weight polysaccharide builder in the cleansing powder composition. In a particularly preferred aspect, 13 to 35% by weight polysaccharide builder is used in the cleansing powder composition.

In an aspect of the present application, a polysaccharide builder used is rice starch and/or a corn starch referred to as INCl: Zea mays (Corn) Starch like, for example, the product sold under the commercial name Farmal™ CS 3757 by Ingredion. In another aspect of the present application, a polysaccharide builder used is unmodified rice starch referred to as INCl: Oryza sativa (rice) starch that is also made commercially available from Ingredion under the Nativacare™ name. In still another aspect of the present application, a polysaccharide builder used is maltodextrin made available as Farmal™ MD 15 by Ingredion. In even another aspect, the polysaccharide builder used is microcrystalline cellulose made available by Active Organics under the name Acticel® 12 name (CAS 9004-34-6).

Desirable sources of cellulose microfibrils include secondary cell wall materials (e.g., wood pulp, cotton), bacterial cellulose, and primary cell wall materials. Preferably, the source of primary cell wall material is selected from parenchymal tissue from fruits, roots, bulbs, tubers, seeds, leaves and combination thereof; more preferably is selected from citrus fruit, tomato fruit, peach fruit, pumpkin fruit, kiwi fruit, apple fruit, mango fruit, sugar beet, beet root, turnip, parsnip, maize, oat, wheat, peas and combinations thereof; and even more preferably is selected from citrus fruit, tomato fruit and combinations thereof. A most preferred source of primary cell wall material is parenchymal tissue from citrus fruit. Citrus fibers, such as those made available by Herbacel® as AQ Plus can also be used as source for cellulose microfibrils. The cellulose sources can be surface modified by any of the known methods including those described in Colloidal Polymer Science, Kalia et al., “Nanofibrillated cellulose: surface modification and potential applications” (2014), Vol 292, Pages 5-31.

Synthetic polymers (non-polysaccharide) may optionally be used as builders in addition to the polysaccharide builders. This category includes crosslinked polyacrylates such as the Carbomers, polyacrylamides such as Sepimax Zen and taurate copolymers such as Aristoflex AVC, the copolymers being identified by respective INCI nomenclature as Sodium Acrylate/Sodium Acryloyldimethyl Taurate and Acryloyl Dimethyltaurate/Vinyl Pyrrolidone Copolymer.

The polysaccharide builder can be present either as generally round or irregular shape particles. Such particles are also desirably hydrophilic. In an aspect of the inventive cleansing powder composition, the particles may have an average particle size from 0.5 to 300 microns, and preferably from 5 to 250 microns, and most preferably, from 10 to 175 microns. Average particle size as used herein means the volume-mean particle size that refers to the diameter of the particle in the aqueous dispersion. For polymer particles that are not spherical, the diameter of the particle is the average of the long and short axes of the particle. Particle sizes can be measured on a Beckman-Coulter LS 13 320 laser-diffraction particle size analyzer, with an art recognized sieve or any other art recognized device.

The present inventors have also surprisingly found that the cleansing powder composition disclosed herein may comprise a higher-than-expected quantity of a viscosity builder as it is conventionally known by one of ordinary skill in the art that increasing inclusion levels of viscosity builders may significantly suppress peak foam volume. Desirable viscosity builders for use in the present cleansing powder compositions include gums and chemically modified cellulosics. Desirable gums include xanthan, sclerotium, pectin, karaya, arabic, agar, guar (including Acacia senegal guar), carrageenan, alginate, Caesalpinia spinosa (i.e., tara gum), and combinations thereof. Desirable chemically modified cellulosics include hydroxypropyl cellulose, hydroxypropyl methylcellulose, ethylcellulose, and sodium carboxy methylcellulose (cellulose gum/carboxymethyl cellulose). In a preferred aspect, the viscosity builder is xanthan gum, sclerotium gum, Caesalpinia Spinosa Gum, hydroxypropyl cellulose, hydroxypropyl methylcellulose, hydroxypropyl starch phosphate, sodium carboxymethyl starch, or a mixture thereof. If included, the inventive composition comprises from 0 to 10%, preferably, 0.1 to 8%, and, more preferably, 0.3 to 5% viscosity builder by weight of the cleansing powder composition.

When using the cleansing powder composition of the present invention, powder and water, in no particular order, can placed in a mixing vessel and shaken, stirred and/or agitated with moderate shear. The resulting end use wash composition can be used as desired (e.g., on hands, face, body, and/or hair). The amount of end use wash composition made is determined by consumer preference. For a single use hand, face, body or hair wash, typically from 0.25 to 12 grams, preferably, from 0.3 to 10 grams, and, most preferably, from 0.5 to 8 grams of cleansing powder composition is used. The amount of water used with the cleansing powder composition to make end use composition is also determined by consumer preference. Often, the amount/weight of water used with cleansing powder composition to produce end use wash composition is from 1 to 10 times, preferably, from 3 to 7 times, and, most preferably, from 4 to 6 times the amount/weight of cleansing powder composition used.

In an embodiment, cleansing powder composition and water, in no particular order, are placed in the hand. Mixing and shearing with both hands results in hand washing and end use wash composition generation simultaneously. End use wash composition for hair washing can be produced in the hands as well. In an embodiment of the invention, water and cleansing composition can be placed in a hand and subsequently supplied to the face or head for face washing or hair and scalp washing where the washing motion provides the shear to make the face wash or shampoo composition as the case may be. In still another embodiment, cleansing powder composition may be applied dry to a substrate like hair, a rug or upholstery that is dry or wet, followed by water (if necessary in the case of prewetted substrate) whereby shear from the hand or an object like a scrub brush results in cleaning and simultaneous generation of cleansing wash composition.

Optional ingredients that may be used in the cleansing powder composition of the present invention include preservatives to assist against the growth of potentially harmful microorganisms when the end use wash composition is made. Suitable traditional preservatives that may be used include hydantoin derivatives, propionate salts, and a variety of quaternary ammonium compounds. Often preferred preservatives are potassium sorbate, iodopropynyl butyl carbamate, phenoxyethanol, methyl paraben, wasabi-based preservatives, propyl paraben, imidazolidinyl urea, sodium dehydroacetate and benzyl alcohol. Especially preferred additives suitable to be employed in the cleansing powder composition of the present invention are 1,2-alkanediols like 1,2-octanediol, 1,2 hexanediol or mixtures thereof. In one aspect, the cleansing powder composition may comprise of a preservative system that is formaldehyde-free, paraben-free, or both.

Traditional fragrance components like eugenol, coumarin, linalyl acetate, citronellal, iris concentrate, terpinyl acetate, terpineol, thymol, pinenes (e.g., alpha and beta pinene) and citronellol may optionally be added to the cleansing powder composition as well.

If employed, the traditional preservatives, vicinal diol and/or fragrance component will not make up more than 2%, and preferably, not more than 1%, and most preferably, from 0.2 to 0.85% by weight of the end use wash composition of the present invention. In an embodiment of this invention from 0.2 to 0.8% by weight optional preservative, vicinal diol and/or fragrance component is used, based on total weight of the end use wash composition. In an embodiment of the invention, no traditional preservative, vicinal diol and/or fragrance component (except for what may be provided in the fragrance used in the end use wash composition) is used in the end use wash composition since such a wash composition can be made and used on demand if desired giving the consumer the option to have a preservative-free wash product.

Other optional ingredients suitable for use include zinc pyrithione, octopirox, or a mixture thereof, especially when the end use wash composition is shampoo that provides antidandruff benefits. Each of these substances may range from 0.05 to 3%, preferably between 0.1 and 2% by weight of the total weight of the end use wash composition.

Additional optional ingredients that may be used include sensory oils and/or exfoliants. Desirable oils include rose, lime, coconut, lavender, argan, sweet almond oil(s) or mixtures thereof. Illustrative exfoliants desirable for use include salt, sugar, apricot, walnut shell, rice, nutmeg and/or oatmeal powder(s). When used, sensory oils and exfoliants can make up from 0.1 to 2% by weight of the end use wash composition, with the proviso that the total amount of fragrance and sensory oil does not exceed 2.5% by weight of the end use composition, and preferably, not more than 2.0 percent by weight of the composition.

The cosmetic powder composition disclosed herein may include vitamins. Illustrative vitamins are Vitamin B₂, Vitamin B₃ (niacinamide), Vitamin B₆, Vitamin C, Vitamin E, Folic Acid and Biotin. Derivatives of the vitamins may also be employed. For instance, Vitamin C derivatives include ascorbyl tetraisopalmitate, magnesium ascorbyl phosphate and ascorbyl glycoside. Derivatives of Vitamin E include tocopheryl acetate, tocopheryl palmitate and tocopheryl linoleate. DL-panthenol and derivatives may also be employed. Total amount of vitamins when present may range from 0.001 to 10%, and preferably from 0.01% to 5%, optimally from 0.1 to 3% by weight of the cosmetic powder composition.

Other optional additives desirable for use include resorcinols like 4-ethyl resorcinol, 4-hexyl resorcinol, 4-phenylethyl resorcinol, dimethoxytoluyl propyl resorcinol, 4-cyclopentyl resorcinol, 4-cyclohexylresorcinol, thiamidol; alpha- and/or beta-hydroxyacids; retinoic acid and its derivatives (e.g., cis and trans); retinal; retinol; retinyl esters such as retinyl acetate, retinyl palmitate, and retinyl propionate; petroselinic acid; conjugated linoleic acid; 12-hydroxystearic acid; mixtures thereof or the like. Still other optional additives like ethanol, quaternary ammonium compounds (like cetrimonium chloride, benzalkonium chloride or the like) and may also be included. Further optional additives including emulsifiers may be used in the cleaving powder composition. Conditioning agents such as polyquaternium compounds (e.g., polyquaternium-67) may also be desirable for inclusion in the inventive composition. Such additives, when used, collectively make up from 0.001 to 3%, preferably, from 0.01 to 2%, and, most preferably, from 0.1 to 1.5% by weight of the end use wash composition.

Desquamation promoters may be present. Illustrative are the alpha-hydroxycarboxylic acids, beta-hydroxycarboxylic acids. The term “acid” is meant to include not only the free acid but also salts and C₁-C₃₀ alkyl or aryl esters thereof and lactones generated from removal of water to form cyclic or linear lactone structures. Representative acids are glycolic and its derivatives, lactic and malic acids. Amounts of these materials when present may range from 0.01 to 3%, and, preferably, from 0.1 to 2% by weight of the end use wash composition.

A variety of herbal extracts may optionally be included in the cosmetic wash compositions of this invention. Illustrative extracts include those removed from green tea, yarrow, ginseng, marigold, hibiscus, ginko biloba, chamomile, licorice, aloe vera, grape seed, citrus unshiu, willow bark, sage and rosemary. Humectants like glycerol and other polyols may also be included. Humectants and/or extracts, such as sorbitol, when used, typically make up from 0.01 to 5%, preferably, from 0.01 to 4%, and, most preferably, from 0.02 to 3% by weight of the end use composition.

Another optional additive suitable for use includes hemp oil with 2.5 to 25% by weight cannabigerol and/or cannabidiol at from 0.5 to 10 percent by weight. When used, such oil makes up from 0.0001 to 12% by weight of the composition, and preferably, from 0.01 to 5% by weight of the end-use composition, including all ranges subsumed therein.

Also optionally suitable for use include materials like chelators (e.g., EDTA), opacifiers (like TiO₂, particle size from 50 to 1200 nm, and preferably, 50 to 350 nm), kaolin, bentonite, zinc oxide, iron oxide, mica, C₈₋₂₂ fatty acid substituted saccharides, lipoic acid, retinoxytrimethylsilane (available from Clariant Corp. under the Silcare 1M-75 trademark), dehydroepiandrosterone (DHEA) or mixtures thereof. Ceramides (including Ceramide 1, Ceramide 3, Ceramide 3B and Ceramide 6) as well as pseudoceramides may also be optionally included as can 10- and/or 12-hydroxystearic acid. Amounts of extract, polyol and these additional materials when used may range from 0.0001 to 3%, and preferably, from 0.001 to 2%, and most preferably, from 0.001 to 1.5% by weight of the end use wash composition.

Colorants or dyes may also be included in the disclosed compositions. These substances may range from 0.05 to 5%, preferably, between 0.1 to 2% by weight of the composition.

Conditioning agents like hydroxypropyltrimonium chloride, 5-ureidohydantoin and/or glyoxyldiureide may be used. The components when used make up from 0.5 to 4%, and, preferably, from 0.75 to 4%, and most preferably, from 1 to 3% by weight of the cleansing powder composition.

Sunscreen actives may also be optionally included in the disclosed cosmetic powder composition. Particularly preferred are such materials as ethylhexyl p-methoxycinnamate, available as Parsol MCX®, Avobenzene, available as Parsol 1789® and benzophenone-3, also known as Oxybenzone. Inorganic sunscreen actives may be employed such as microfine titanium dioxide, zinc oxide, polyethylene and various other polymers. Amounts of the sunscreen agents when present may generally range from 0.01 to 3%, preferably, from 0.5 to 2%, optimally, from 0.75 to 1.5% by weight of the end use wash composition.

Conventional buffers/pH modifiers may be used. These include commonly employed additives like sodium hydroxide, potassium hydroxide, hydrochloric acid, citric acid/citrate buffers, triethanolamine, or mixtures thereof. These materials are added at amounts to obtain the desired pH of the end use wash composition. Upon desired hydration, it is expected that the pH of the cleansing powder composition will range from 3.5 to 7.5, preferably, from 4.0 to 6.5, and, most preferably, from 4.5 to 5.5 where pH is determined using a Thermo Fisher Scientific pH meter.

In an embodiment of the invention, the end use wash composition of the present invention is preferably free of sulfate, and therefore, having less than 2.5%, preferably, less than 1.5%, and, most preferably, less than 0.5% by weight of a sulfate. In an especially preferred embodiment, the end use wash composition has no (0% by weight) sulfate comprising component.

The viscosity of the end use wash composition is typically from 2,000 to 70,000 cPs, preferably, from 3,500 to 50,000 cPs, and, more preferably, from 3,000 to 30,000 cPs. In another aspect, the viscosity of the end use composition is from 3,500 to 62,500 cPs. Viscosity may be measured with art recognized instrumentation such as a Discovery HR-2 Hybrid Rheometer for 30 seconds at 25° C. and 4/s, where the end use wash composition was prepared via diluting 1 part of cleansing powder to 3 parts of water.

When preparing cleansing powder composition, ingredients may be mixed and/or agitated at atmospheric pressure. The temperature at which mixing and agitation occurs is typically from 20° C. to 50° C. Mixing and/or agitation is stopped when a homogeneous mixture is obtained and the resulting cleansing powder composition is free of aggregated particles. The particles of the cleansing powder composition will have an average particle size from 0.5 to 300 microns, and preferably from 5 to 250 microns, and most preferably, from 10 to 175 microns as determined with art recognized devices as noted herein.

A wide variety of packaging can be employed to store the cleansing powder composition of this invention. Jars, sachets, shakers, bags, pumps, bottles, metallic containers as well as plastic containers may be used. Preferably, the packaging used with the cleansing powder composition of the present invention is packaging made from recycled material like post-consumer resins or biodegradable material. Most preferably, the cleansing powder composition is sold in biodegradable packaging material, like paper or cardboard material, whereby the jar or bottle, as the case may be, is refilled with cleansing powder composition sold in biodegradable packaging. In an especially preferred embodiment, the cleansing powder composition is used by the consumer directly from the biodegradable packaging.

### EXAMPLES

The following examples are provided to facilitate an understanding of the present invention. The examples are not intended to limit the scope of the claims.

### Example 1: Sample Formulations

Cleansing powder compositions were made according to the present invention with the ingredients and amounts set forth in Table I as two different formulation samples. The ingredients were combined via agitation at atmospheric pressure and at 25° C. Surprisingly, no visual clumping or agglomeration of composition was observed even after the product was stored at one week under representative hot and humid conditions (75% RH at 40° C.) and under representative high humidity (80% RH at 25° C.). Additionally, the cleansing powder compositions had a bulk density of about 0.55 g/cm³ and was easily flowable in the absence of visual static cohesion.

Weight Percent (wt %)

| Ingredient Name | Formula A | Formula B |
|---|---|---|
| Sodium bicarbonate | 15 | 18 |
| Sodium sesquicarbonate | 3.5 | 3.5 |
| Citric acid | 16 | 16.3 |
| Sodium Cocoyl Isethionate | 24 | 26.1 |
| Viscosity builder | 2.0 | 0.0-4.0 |
| Polyquaternium-67 | 0.5 | 0.0 |
| Humectant | 3.0 | 3.0 |
| Chelator | 0.1 | 0.1 |
| Preservative | 0.6 | 0.6 |
| Tricalcium citrate | 5.0 | 5.0 |
| Microcrystalline Cellulose | — | Balance |
| Zea mays (Corn) starch | 4.7 | — |
| Maltodextrin | 25 | — |
| Fragrance and benefit agent | 0.6 | 0.6 |
| | 100.0 | 100.0 |

About one (1) gram of cleansing powder composition described in the table presented in Example 1 were applied to the left forearm of trained panelists followed by about four (4) grams of water. The panelists, using moderate shear with their right hands were able to mix the water and cleansing powder composition to generate an end use wash composition that was not grainy and that provided consumer acceptable (creamy) lathering when being used. The flowability of the powder made it easy to remove from packaging with an open orifice and a biodegradable sachet or pouch. Prior to use by the panelists, the cleansing powder compositions made according to the inventive compositions were assessed and confirmed to be easily hydratable and easily dispensed as free-flowing powders.

### Example 2: Viscosity and Self-Foaming Capability while Varying Viscosity Builder Identity and Concentrations

The ingredients of the formulae made in Example 2 were mixed in a manner similar to the one described in Example I.

| | | | | Viscosity |
|---|---|---|---|---|
| | | Weight | | (4/s, 30 s @ |
| | | Percent of | | 25° C.) in |
| | Viscosity Builder | viscosity | Peak Foam | centipoise |
| Formula | Ingredient | builder (wt %) | Volume (mL) | (cPs) |
| Formula A | Sodium carboxymethyl | 2.0 | 27 | 60360 |
| | starch | | | |
| | Hydroxypropyl Starch | 2.0 | 27 | 52700 |
| | Phosphate | | | |
| | Hydroxypropyl | 2.0 | 24 | 7717 |
| | Methylcellulose | | | |
| | Sclerotium Gum | 2.0 | 30 | 9325 |
| Formula B | Xanthan Gum | 0.0 | 37 | 6880 |
| | | 0.5 | 35 | 9239 |
| | | 1.0 | 35 | 9418 |
| | | 2.0 | 32 | 27630 |
| | | 4.0 | 30 | 28440 |
| | Caesalpinia Spinosa | 2.0 | 32 | 15830 |
| | Gum | | | |
| | Hydroxypropylcellulose | 2.0 | 34 | 13710 |
| Bioré ® Baking Soda | Sodium Carboxymethyl | Unknown | 17.0 | 1394 |
| Cleansing Scrub | Starch, Silica | | | |
| DHC Face Wash | Silica | Unknown | 2.0 | Too thin to |
| Powder | | | | measure |
| OLAY ® Exfoliating | Hydroxypropyl | Unknown | 9.0 | Too thin to |
| Face & Body Powder | methylcellulose, silica | | | measure |

Viscosity was measured using a rheometer (i.e., Discovery HR-2 Hybrid Rheometer with a 40 mm sandblasted plate) at a setting of 4/s for 30 s @ 25° C. In preparation for viscosity measurements, the desired sample was diluted (1 part cleansing powder composition to 3 parts water). Samples were allowed to sit overnight before viscosity measurements were conducted.

Peak foam volume was another criterion of particular interest to the present inventors. Foam volume was measured by placing 1 gram (g) of cleansing powder composition in a 100 milliliter (mL) graduated cylinder. 9 g of water was added to 1 g of cleansing powder composition without subjecting said cleansing powder composition to any shear or agitation. The reaction of dry acidic and dry alkaline material in water leads to formation of carbon dioxide gas that instantly triggers the hydrated composition to self-foam in the inventive composition. Peak foam volume was measured in mL as the unit. 1 g of cleansing powder composition and 9 g of water were subsequently detracted from the peak foam volume measurements obtained. All measurements as currently presented in this Example already account for such a subtraction.

An end use wash composition is considered to possess superior self-foaming capabilities when the resulting peak foam volume exceeds 20 mL as per the aforementioned methodology. Viscosity greater than 3,500 centipoise (cPs) is also correlated with a superior consumer use experience.

It was unexpectedly determined that cleansing powder compositions made according to the inventive composition disclosed herein were able to tolerate high amounts of viscosity builder without resulting in significant compromises in terms of peak foam volume, which would have been conventionally expected. For instance, in this particular Example, in-scope compositions were able to have 4% xanthan gum by weight while still producing excellent foam volume. It is also noted that inclusion of such viscosity builders does not negatively impact the flowability of the cleansing powder composition, that is to say that the composition remained free of agglomeration (i.e., was free-flowing).

The viscosity and peak foam volume of several competitor cleansing powder products for facial and body use commercially available on the market were also measured and it is observed that such powdered products did not produce desirable peak foam volume or viscosity upon dilution. In contrast, cleansing powder compositions according to the present application were able to deliver such benefits even when the identity and/or amount of viscosity builder was modified.

### Example 3: Cleansing Powder Compositions Varying Polysaccharide Builder, Carboxylic Acid Salt, and Amounts Thereof

| | | Weight percent (wt %) | | | | | | | |
|---|---|---|---|---|---|---|---|---|---|
| Function | Ingredient | 1 | 2 | 3 | 4 | 5 | 6 | 7 | 8 |
| Self- | Sodium | 15 | 15 | 15 | 15 | 15 | 15 | 24 | 28 |
| foaming | bicarbonate | | | | | | | | |
| agent | Citric Acid | 16 | 16 | 16 | 16 | 16 | 16 | 25 | 28 |
| | Sodium | 3.5 | 3.5 | 3.5 | 3.5 | 3.5 | 3.5 | 3.5 | — |
| | Sesquicarbonate | | | | | | | | |
| Cleansing | Sodium Cocoyl | Balance | Balance | Balance | Balance | Balance | Balance | Balance | Balance |
| surfactant | Isethionate | | | | | | | | |
| Stabilizing | Zea mays (Corn) | — | — | — | — | 5.0 | 5.0 | 7.0 | — |
| system | Starch | | | | | | | | |
| materials | Microcrystalline | — | — | 30 | 25 | — | — | — | — |
| | cellulose | | | | | | | | |
| | Maltodextrin | 30 | 25 | — | — | 25 | 25 | 7.0 | — |
| | Tricalcium citrate | — | 5.0 | — | 5.0 | — | — | — | 5.0 |
| | Trimagnesium | — | — | — | — | — | 5.0 | 5.0 | — |
| | citrate | | | | | | | | |
| Viscosity | Xanthan Gum | 2.0 | 2.0 | 2.0 | 2.0 | 2.0 | 2.0 | 2.0 | 2.0 |
| Builder | | | | | | | | | |

| | | | | | | | | |
|---|---|---|---|---|---|---|---|---|
| Humectant | 3.0 | 3.0 | 3.0 | 3.0 | 3.0 | 3.0 | 3.0 | — |
| Benefit Agent | 0.5 | 0.5 | 0.5 | 0.5 | 0.5 | 0.5 | 0.5 | — |
| Preservative | 0.6 | 0.6 | 0.6 | 0.6 | 0.6 | 0.6 | 0.6 | 1.0 |
| Fragrance | 0.6 | 0.6 | 0.6 | 0.6 | 0.6 | 0.6 | 0.6 | — |
| Stable at 75% RH @ 40° C. | no | yes | no | yes | no | yes | yes | no |
| (hot and humid)? | | | | | | | | |

Example 3 unexpectedly demonstrates that when compositions are made consistent with the disclosed cleansing powder composition, optimal stability of the inventive effervescent cleansing powder compositions is observed. The ingredients of the formulae made in this Example were mixed in a manner similar to the ones described in Example 1. The samples in this Example show that mere inclusion of a polysaccharide builder alone is not sufficient for a stable cleansing powder composition at conditions of 75% RH at 40° C., but that once a carboxylic acid salt is added according to the present application, desired stability is immediately achieved.

Similarly, Sample 8 has only a carboxylic acid salt (i.e., absence of polysaccharide builder) and one observes that it is not found to be stable under the specified hot and humid conditions, again illustrating that a composition having only a salt of carboxylic acid without a polysaccharide builder does not give the stabilizing benefit observed when both components are contained.

### Example 4: Sample Formulations not In-Scope of Present Application

| | | Weight percent (wt %) | | | | | |
|---|---|---|---|---|---|---|---|
| Function | Ingredient | 9 | 10 | 11 | 12 | 13 | 14 |
| Self- | Sodium Bicarbonate | 14 | 29 | 28 | 35 | 45 | 42 |
| foaming | Citric Acid | 14 | 28 | 28 | 28 | 30 | 42 |
| agent | Sodium Sesquicarbonate | — | 2.5 | — | — | — | — |
| Cleansing | Sodium Cocoyl Isethionate | Balance | Balance | Balance | — | — | — |
| surfactant | Cocamidopropyl Betaine | 7.5 | — | — | — | — | 15.0 |
| | Disodium Lauryl | — | — | — | 16 | — | — |
| | Sulfosuccinate | | | | | | |
| | Sodium Lauroyl Glutamate | — | — | — | — | 9.0 | — |
| Stabilizing | Tapioca Starch | — | — | — | — | — | — |
| System | Oryza Sativa (Rice) Starch | — | 5.5 | — | — | — | — |
| materials | Maltodextrin | 30 | — | — | — | — | — |
| | Hydroxypropyl Starch | — | — | — | 8.5 | 5.5 | — |
| | Phosphate | | | | | | |
| | Sodium Carboxymethyl | — | — | — | 8.5 | 5.5 | — |
| | Starch | | | | | | |
| | Calcium citrate | — | — | 5.0 | — | — | — |
| | Magnesium Aluminum | — | 2.0 | — | — | — | — |
| | Silicate | | | | | | |
| Viscosity | Xanthan Gum | 7.5 | — | 2.0 | — | — | — |
| Builder | | | | | | | |

| | | | | | | |
|---|---|---|---|---|---|---|
| Benefit Agent | 8.8 | 0.03 | 0.03 | 4.0 | 5.0 | — |
| Preservative | 0.5 | 0.9 | 1.0 | — | — | — |
| Fragrance | 0.6 | 0.8 | — | — | — | 1.0 |

All samples in Example 4 failed stability after 1 week under high humidity and high temperature conditions (i.e., 75% RH at 40° C.). Products were found to have settled/caked and also lost free-flowability. The ingredients of the formulae made in this Example were mixed in a manner similar to the ones described in Example 1. None of the formulae made in this Example were made with ingredients consistent with those of the inventive cleansing powder compositions. The formulations not made according to this invention were not usable as it agglomerated and/or settled/caked at 75% RH at 40° C. conditions.

### Example 5: Cleansing Powder Compositions Varying Surfactants

Weight percent (wt %)

| Ingredient | 15 | 16 | 17 |
|---|---|---|---|
| Sodium bicarbonate | 18 | 18 | 18 |
| Citric Acid | 16 | 16 | 16 |
| Sodium Cocoyl Isethionate | 19 | 19 | 19 |
| Cocamidopropyl Betaine | 7.0 | — | — |
| Sodium Methyl Cocoyl Taurate | — | 7.0 | — |
| Sodium Lauryl Sulfate | — | — | 7.0 |
| Zea mays (Corn) Starch | 5.0 | 5.0 | 5.0 |
| Microcrystalline cellulose | Balance | Balance | Balance |
| Tricalcium citrate | 5.0 | 5.0 | 5.0 |
| Xanthan Gum | 2.0 | 2.0 | 2.0 |
| Benefit Agent | 0.03 | 0.03 | 0.03 |
| Preservative | 1.0 | 1.0 | 1.0 |
| Fragrance | 0.6 | 0.6 | 0.6 |

Example 5 shows that the solid surfactant system comprising isethionate may also be used in combination with a variety of other surfactants, such as cocamidopropyl betaine, sodium methyl cocoyl taurate, and sodium lauryl sulfate as presented here as long as such additional surfactants are in accordance to the inventive composition. All cleansing powder composition samples in this Example passed 1 week stability and meet the viscosity and foam volume criteria as specified by the present application (i.e., peak foam volume exceeds 20 mL and viscosity is greater than 3,500 cPs, respectively). The ingredients of the formulae made in this Example were mixed in a manner similar to the ones described in Example 1.

1. A cleansing powder composition comprising:
a. a solid surfactant system comprising an isethionate, taurate, or mixture thereof;
b. an effervescent system comprising a dry acidic material having an acid dissociation constant (pKₐ) from 2 to 12, and a logP value less than 0, and a dry alkaline material;
c. a stabilizing system comprising a polysaccharide builder and a salt of carboxylic acid; and
d. optionally a viscosity builder;
wherein the cleansing powder composition is anhydrous,
wherein the salt of carboxylic acid is an alkali citrate, an alkaline citrate, or a mixture thereof comprising tricalcium citrate, trimagnesium citrate, trisodium citrate, monosodium citrate, and mixtures thereof in amounts ranging from 1 to 20% by weight of the salt of carboxylic acid.

2. The cleansing powder composition according to claim 1, wherein the solid surfactant system comprises an isethionate, a taurate, or a mixture thereof, in amounts ranging from 5 to 40% by weight of the cleansing powder composition.

3. The cleansing powder composition according to claim 1, wherein the solid surfactant system comprises sodium cocoyl isethionate, cocamidopropyl betaine, sodium methyl cocoyl taurate, sodium lauryl sulfate, sodium lauroyl glutamate, sodium cocoyl glutamate, sodium cocoyl glycinate, disodium lauryl sulfosuccinate, or mixtures thereof.

4. The cleansing powder composition according to claim 1, wherein the dry acidic material comprises 5 to 35% citric acid by weight of the cleansing powder composition.

5. The cleansing powder composition according to claim 1, wherein the dry alkaline material comprises sodium bicarbonate, potassium bicarbonate, sodium sesquicarbonate, or a mixture thereof and is present in amounts ranging from 5 to 35% dry alkaline material by weight of the cleansing powder composition.

6. The cleansing powder composition according to claim 1, wherein the dry acidic material to dry alkaline material has a weight ratio in the cleansing powder composition of 30:70 to 70:30.

7. The cleansing powder composition according to claim 1, wherein the polysaccharide builder comprises microcrystalline cellulose, maltodextrin, Zea mays (corn) starch, tapioca starch, or a mixture thereof in amounts ranging from 8 to 60% polysaccharide builder by weight of the cleansing powder composition.

8. The cleansing powder composition according to claim 1, wherein the viscosity builder comprises xanthan gum, sclerotium gum, Caesalpinia Spinosa Gum, hydroxypropyl cellulose, hydroxypropyl methylcellulose, hydroxypropyl starch phosphate, sodium carboxymethyl starch, or mixtures thereof in amounts ranging from 0 to 10% viscosity builder by weight of the cleansing powder composition.

9. The cleansing powder composition according to claim 1, wherein the composition is a precursor to a hands, face, body, hair, nails, or inanimate object wash composition.

10. The cleansing powder composition according to claim 1, wherein the cleansing powder composition is stable and does not agglomerate under conditions of high humidity and high temperature (75% relative humidity at 40° C.) for at least 7 days.

11. The cleansing powder composition according to claim 1, wherein the cleansing powder composition is in the form of a free-flowing powder easily dispensable through a small orifice.

12. The cleansing powder composition according to claim 1, wherein the composition instantly effervesces (self-foams) upon hydration with water, producing a minimum of 20 ml of peak foam volume and further wherein water is present at a weight that is from 1 to 10 times the weight of the cleansing powder composition used to make a wash composition.

13. A process of manufacturing the wash composition comprising the step of hydrating with water the cleansing powder composition according to claim 12.

14. A method for washing comprising the steps of:
hydrating the cleansing powder composition according to claim 1 to produce a wash composition; and
b. contacting skin, hair, nails, or an inanimate object with the wash composition,
wherein the cleansing powder composition is hydrated with shear to produce the wash composition before or after contact with the skin, hair, nails, or an inanimate object.

15. The cleansing powder composition according to claim 1, wherein the dry acidic material has an acid dissociation constant (pKₐ) from 2 to 8, and a logP value from −2 to −0.05, and further wherein the dry acidic material comprises 7 to 32% citric acid by weight of the cleansing powder composition.

16. The cleansing powder composition according to claim 1, wherein the salt of carboxylic acid is present in amounts ranging from 1.5 to 15% by weight of the salt of carboxylic acid.

17. The cleansing powder composition according to claim 1, wherein the dry acidic material to dry alkaline material has a weight ratio in the cleansing powder composition of 40:60 to 60:40.

18. The cleansing powder composition according to claim 1, wherein the dry acidic material to dry alkaline material has a weight ratio in the cleansing powder composition of 45:55 to 55:45.

19. The cleansing powder composition according to claim 1, wherein the polysaccharide builder is present in amounts ranging from 10 to 50% polysaccharide builder by weight of the cleansing powder composition.

20. The cleansing powder composition according to claim 1, wherein the viscosity builder is present in amounts ranging from 0.1 to 8% viscosity builder by weight of the cleansing powder composition.
